(19) United States
(12) Patent Application Publication
(10) Pub. No.: US 2026/0355093 A1
(43) Pub. Date: Nov. 12, 2026

(54) CELLULOSIC NANOFIBERS AND FORMULATIONS DERIVED THEREFROM

(71) Applicant: Macro Oceans Inc., San Francisco, CA (US)

(72) Inventor: Colin Hepburn, San Francisco, CA (US)

(73) Assignee: Macro Oceans Inc., San Francisco, CA (US)

(21) Appl. No.: 19/675,126

(22) Filed: May 12, 2026

Related U.S. Application Data
(60) Provisional application No. 63/804,403, filed on May 12, 2025.

(51) Int. Cl. A61K 8/73; A61K 8/02; A61K 8/27; A61K 8/29; A61Q 17/04

(52) U.S. Cl. CPC A61K 8/731; A61K 8/027; A61K 8/27; A61K 8/29; A61Q 17/04

(57) ABSTRACT

A formulation can include nanofibers, an optional solvent, and additive(s). The nanofibers can be cellulosic fibers derived from (e.g., predominantly derived from, including only, including essentially only, etc.) natural product(s), such as seaweed. The nanofibers can have a width (e.g., diameter) between 1 and 10 nm and a length at least approximately 300 times greater than the width.

### CROSS-REFERENCE TO RELATED APPLICATION

This application claims the benefit of U.S. Provisional Application No. 63/804,403, filed 12 May 2025, which is incorporated in its entirety by this reference.

### TECHNICAL FIELD

This invention relates generally to the health and wellness field, and more specifically to a new and useful formulations in the health and wellness field.

### BRIEF DESCRIPTION OF THE FIGURES

FIG. 1 is an illustrative example of the technology.

FIG. 2 is a diagram of a variant of the nanofiber.

FIG. 3A is a micrograph of an example of cellulosic nanofibers synthesized from seaweed.

FIG. 3B is a micrograph of an example of cellulosic nanofibers synthesized from wood pulp.

FIG. 4 shows plots of viscosity vs shear rate, viscosity vs shear stress, and storage and loss modulus for exemplary cellulosic nanofibers (CNF) samples, exemplary modified cellulosic nanofibers (mCNF), exemplary formulations that include a mixture of unmodified and modified cellulosic nanofibers (CNF/mCNF) as well as comparison data with guar gum and xanthan gum examples.

FIG. 5 shows plots of viscosity vs shear stress, comparing an exemplary formulation with 0.5% nanofiber content wherein the nanofibers are fully functionalized to achieve a maximum charge density (e.g., 100% charge density) and formulations with 1% guar gum and xanthan gum.

FIG. 6 is a plot showing the water absorption of variants of the cellulosic nanofiber.

FIG. 7 show micrographs of variants of the cellulosic nanofiber (e.g., varying number of passes through a homogenizer) at different temperatures (65 °C, 75 °C, and 85 °C) and histograms showing corresponding fiber widths.

FIGS. 8A, 8B, 8C, and 8D, show rheological data for variants of the cellulosic nanofibers (e.g., varying number of passes through a homogenizer) at different temperatures (65 °C, 75 °C, and 85°). FIG. 8D shows an exemplary relationship between storage modulus and the number of passes through a homogenizer.

FIG. 9 show plots of transmittance vs wavelength for variants of the cellulosic nanofiber (e.g., varying number of passes through a homogenizer) at different temperatures (65 °C, 75 °C, and 85°).

FIG. 10 show a diffractogram and crystallinity indices of variants of the cellulosic nanofiber.

FIG. 11A show viscosity vs shear rate of variants of the cellulosic nanofiber with varying charge density (0%, 20%, 100%). FIG. 11B show viscosity vs shear rate of a variant of the nanofiber and carbomer at concentrations of 0.5 wt %.

### DETAILED DESCRIPTION

The following description of the embodiments of the invention is not intended to limit the invention to these embodiments, but rather to enable any person skilled in the art to make and use this invention.

### 1. Overview

A formulation can include: cellulosic nanofibers 100, an optional solvent(s) 200, and additive(s) 300. The formulation preferably leverages materials derived from (e.g., predominantly derived from, including only, including essentially only, etc.) natural product(s). The formulation can be used for beauty, health, and/or other personal care products.

Exemplary applications of the formulations include: skincare products (e.g., lotion, cream, gel, serum, sunscreen, diaper cream, etc.), cosmetics (e.g., foundation, primer, eyeliner, mascara, lipstick, etc.), personal care products (e.g., toothpaste, hand sanitizer, shampoo, conditioner, hair gel, hair styling product, personal lubricant, diapers, sanitary pads, tampons, etc.), pharmaceuticals (e.g., topical medications, eye drops, wound care gels, etc.), or household products (e.g., cleaners, soaps, detergents, etc.). However, variations of the formulation can be used for other applications.

### 2. Examples

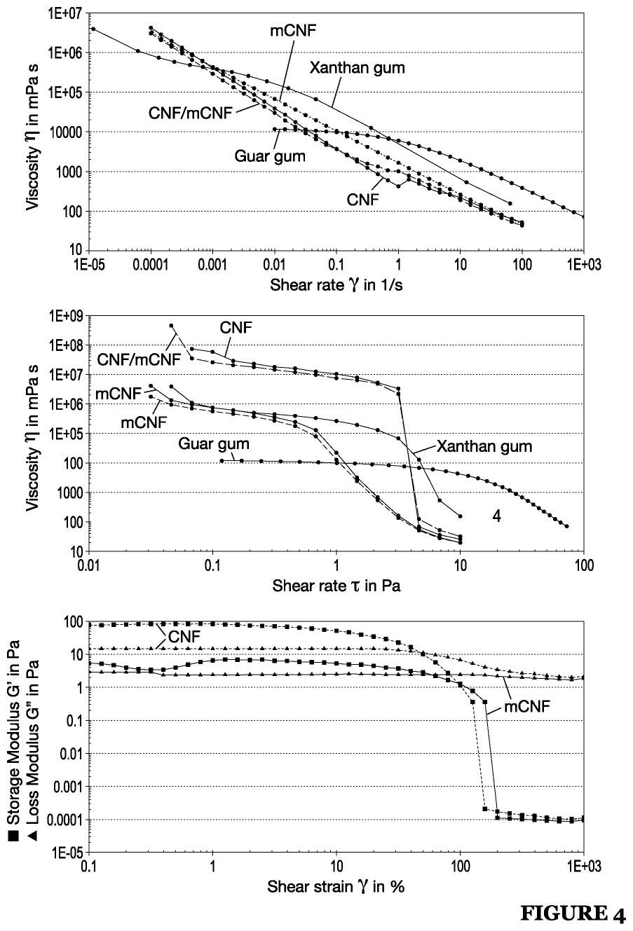

An illustrative example of the formulation includes nanocellulose derived from seaweed, where the nanocellulose is made from nanofibers with a width (e.g., diameter) between 6 and 10 nm and a length at least approximately 300 times greater than the width. The nanocellulose can be hydrophilic, hydrophobic, and/or amphiphilic (e.g., based on a surface modification of the nanofibers). The nanocellulose can have a water absorption capacity greater than or equal to 500 g water per 1 g of nanofiber. Aqueous suspensions of the nanofiber can exhibit rheological behaviors like shear thinning, wherein viscosity decreases with increasing shear rate (as evidenced by a linear viscosity/shear rate curves as shown for example in FIG. 4). In some variants, the viscosity breaks at low shear stress, allowing the material to flow readily under small applied force. These rheological properties can result in favorable organoleptic characteristics of the suspension (e.g., a waterlike and/or non-viscous skin feel). In some variations (e.g., for sunscreen applications), The nanofiber can have an elastic modulus and/or yield stress that allows for the suspension of particles (e.g. zinc oxide) in aqueous dispersions.

### 3. Technical Advantages

Variants of the technology can confer one or more advantages over conventional technologies.

First, the inventors have discovered that cellulose nanofibers (particularly those extracted from algae) can exhibit a water absorption capacity of at least 500 times their own weight in water. This high water absorption can facilitate the formation of aqueous dispersions with reduced material input (compared to other natural ingredients), making the nanofiber efficient in hydration-based formulations (such as creams, gels, serums). This water absorption capacity can further contribute to improved organoleptic properties in personal care products (such as enhanced moisturization and smoother texture).

Second, variants of the nanofiber technology can provide a desirable topical experience due to the low yield stress exhibited in aqueous formulations, such that viscosity decreases when shear is applied during spreading. The formulation can exhibit relatively low viscosity under shear. The nanofiber dispersions can display shear-thinning behavior with a water-like consistency under gentle stress (e.g., finger application). This behavior can result in highly spreadable, fast-absorbing formulations ideal for applications including but not limited to cosmetics, dermatologicals, or pharmaceuticals.

Third, the nanofibers can have improved suspension capabilities compared to other nanocellulose fibers. The nanofibers have diameters in the 6-10 nanometer range and lengths at least 300 times their diameter which can contributes to its ability to form a suspension in aqueous and non-aqueous solvents and/or to suspend particles such as mineral oxides (e.g., zinc oxide, titanium dioxide) in low-viscosity dispersions. As such, the nanofiber can serve as a stabilizing agent in formulations that require uniform particle suspension (e.g., without relying on high viscosity or synthetic thickeners).

Fourth, variants of the nanofiber can exhibit high physicochemical stability across a broad range of conditions. For example, the nanofiber can maintain structural integrity under both acidic and alkaline pH levels (e.g., pH 3-12), extreme temperatures (e.g., temperatures elevated above and/or depressed below room temperature such as encountered during formulation, manufacturing, packaging, shipment, storage, etc.). The nanofibers can also be chemically stable in the presence of salts, electrolytes, and/or oxides, which can be included in active formulations.

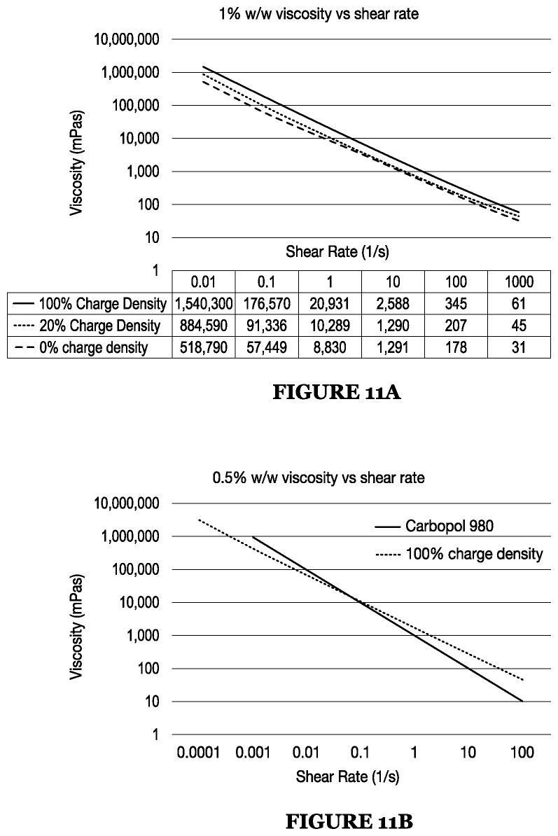

Fifth, variants of the nanofiber can be surface-modified to introduce specific functionalities, such as hydrophilic, hydrophobic, and/or amphiphilic characteristics. These modifications can enable tunable performance properties including emulsification, suspension behavior, controlled cargo release, and other properties. As a result, the nanofiber can be tailored for diverse applications ranging from topical drug delivery to emulsions and hydrogels. In some variants, surface modification can further introduce or alter charged functional groups on surfaces of the nanofibers, thereby modifying a surface charge density of the nanofibers. In variants, adjusting the charge density can influence the behavior of nanofiber dispersions, as shown for example in FIG. 11A. In some variants, increased surface charge density can promote electrostatic repulsion between fibers, which can enhance swelling and dispersion of the nanofibers. In variants, these interactions can influence rheological properties such as viscosity, yield stress, viscoelastic behavior, and shear-thinning response of nanofiber suspensions. The degree and type of surface functionalization can provide a mechanism for tuning rheological performance of formulations comprising the nanofibers. This can allow formulations to be engineered for specific flow, suspension, or delivery characteristics depending on the intended application.

Sixth, the nanofiber can be more sustainable and/or more environmentally than its alternatives. For instance, the nanofiber can be derived from natural sources (such as algae or seaweed) and does not rely on petrochemical inputs. Thus, the nanofiber(s) can provide a sustainable, plant-based alternative to synthetic polymers like carbomer, while maintaining comparable functional properties. Accordingly, the nanofiber can offer an eco-conscious solution aligned with market demands for renewable, biodegradable, and/or clean-label ingredients. Additionally, in certain embodiments, the nanofiber can be produced using relatively low-energy methods, such as mechanical fibrillation or enzymatic pretreatment. Such techniques can provide an energetically efficient alternative to traditional polymer synthesis processes, which often require high temperatures, organic solvents, and/or hazardous reagents. In some variants, the nanofiber formulations can provide an alternative to synthetic polymer rheology modifiers used in personal care and cosmetic products. For example, synthetic polymers such as carbomers and other microplastic-derived materials may be increasingly subject to regulatory restrictions. As such, nanofibers derived from natural sources (e.g., seaweed-derived cellulose nanofibers) can provide a naturally derived alternative capable of delivering comparable rheological and suspension properties while aligning with sustainability demands.

However, further advantages can be provided by the system and method disclosed herein.

### 4. System

A formulation can include: nanofibers 100, an optional solvent(s) 200, and additive(s) 300. The formulation preferably leverages materials derived from (e.g., predominantly derived from, including only, including essentially only, etc.) natural product(s). The formulation can be used for beauty, health, and/or other personal care products.

Exemplary applications of the formulations include: skincare products (e.g., lotion, cream, gel, serum, sunscreen, etc.), cosmetics (e.g., foundation, primer, eyeliner, mascara, lipstick, etc.), personal care products (e.g., toothpaste, hand sanitizer, shampoo, conditioner, hair gel, hair styling product, personal lubricant, diapers, sanitary pads, tampons, etc.), pharmaceuticals (e.g., topical medications, eye drops, wound care gels, etc.), or household products (e.g., cleaners, soaps, detergents, etc.). However, variations of the formulation can be used for other applications. In a first example, the nanofiber can be used in a face cream as a means for improving the cream's texture and/or as a means for increasing water content in the top layers of the skin upon application. In a second example, the nanofiber can be used in diapers as a means for absorbing moisture. In a third example, the nanofiber can be used in a spray on sunscreen (where the nanofibers preferably do not clog the nozzle of the spray).

The formulation can be a gel, cream, paste, ointment, lotion, liquid, aerosol, spray, foam, powder, and/or any other formulation type.

The nanofiber(s) can function to act as a suspension agent, thickening agent, gelling agent, emulsifier, humectant, absorbent, waterproofing agent, and/or can otherwise function in a formulation. The nanofiber(s) are preferably a natural product ingredient. However, the nanofiber can be derived from (e.g., a modified from) a natural product and/or may include (e.g., form a copolymer with) artificial products.

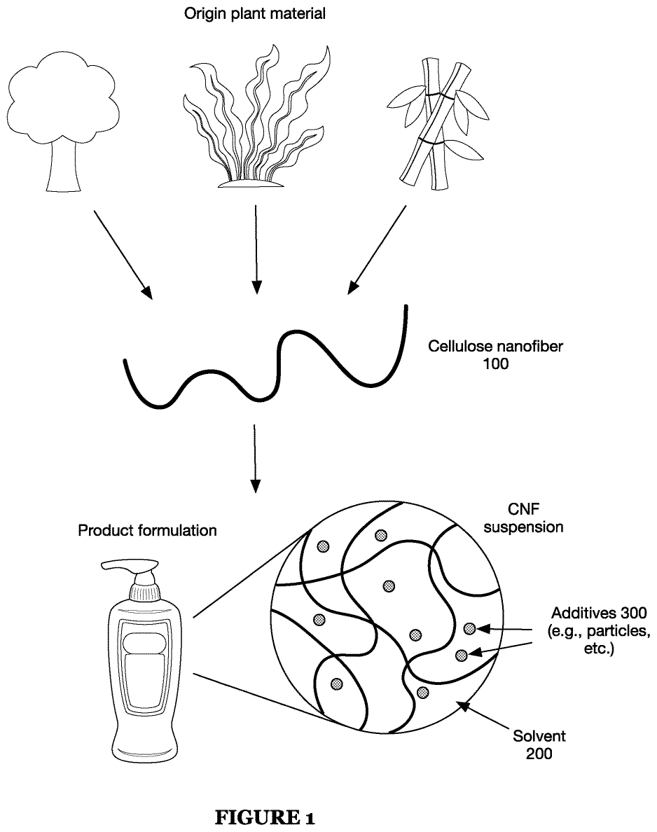

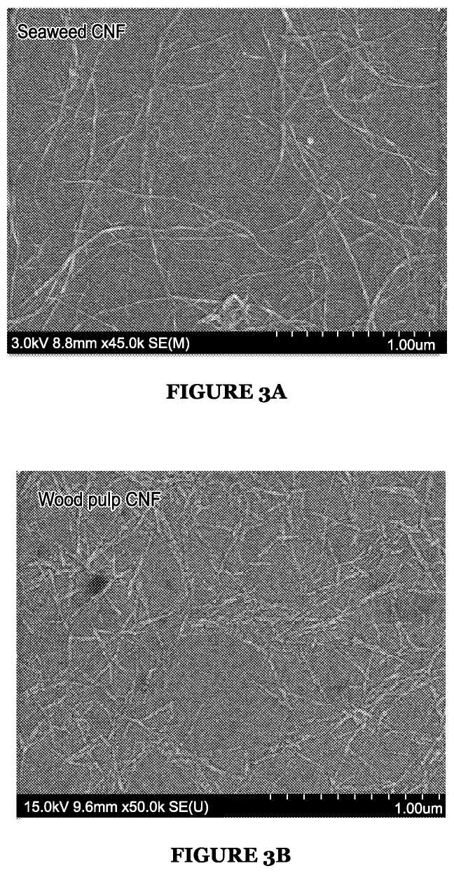

As shown for example in FIG. 1, the nanofiber(s) 100 (NF) can be derived from plants. In a first variant, the nanofiber is preferably derived from seaweed (e.g., green seaweed, red seaweed, brown seaweed, etc.) and/or algae (e.g., macroalgae, microalgae, kelp, etc.). In an example, the nanofiber can be recovered from green seaweeds (e.g., Ulva lactuca, Codium fragile, Cladophora spp., etc.), brown seaweeds (e.g., Macrocystis pyrifera, laminaria spp., Fucus vesiculosus, sargassum spp., Ecklonia maxima, etc.), or red seaweeds (e.g. Eucheuma spp., Chondrus crispus, Porphyra spp., Gracilaria spp., Gelidium spp., etc.). The inventors have found that variants of the nanofibers synthesized from seaweed can have desirable chemical and/or physical properties (e.g. hydrophilicity, large aspect ratio, etc.) that make them suitable for a range of functions in products. In a second variant, the nanofiber can be derived from wood (e.g., wood pulp, softwood, hardwood, etc.), bamboo, cotton, hemp, flax, stalks (e.g., corn), straw (e.g., wheat), husks (e.g., rice, coconut, corn, etc.), and/or any other plant-based materials. As shown for example by comparing FIG. 3A and FIG. 3B, the nanofibers derived from seaweed can have different physical properties from those derived from other (typically woody) plants. The inventors have found the properties of nanofibers derived from seaweed to be particularly beneficial for formulations. In variants, nanofibers derived from macroalgae cellulose can exhibit structural and functional improvements compared to nanofibers derived from wood cellulose. For example, macroalgae-derived cellulose can contain a higher proportion of cellulose Ia crystalline structure and can produce longer fibers and/or fibrils during fibrillation processes. For example, macroalgae-derived cellulose can contain a higher proportion of cellulose Ia crystalline structure (e.g., about 60-80% Ia, compared to about 40-60% for wood-derived cellulose) and can produce longer fibers and/or fibrils during fibrillation processes (e.g., in variants, an average fibril lengths of about 1-5 μm, compared to about 0.5-2 μm for wood-derived nanofibers). These structural differences can contribute to higher storage moduli, improved network formation, and enhanced suspension capability in aqueous formulations

The nanofibers 100 are preferably made of beta-glucan based polysaccharides (e.g., cellulose, α-cellulose, β-cellulose, etc.). The nanofibers preferably do not include significant quantities of (e.g., less than about 5 wt %) hemicellulose (e.g., xyloglucans, xylans, mannans, glucomannans, β-(1→3,1→4)-glucans, etc.), phenylpropanoids (e.g., lignin, lignan, suberin, chalcones, monolignols, etc.), chitosan, dextrin, dextran, fructan, galactan, manan, pectin, amylopectin, xanthan gum, and/or other biopolymers. This exclusion (e.g., inclusion at amounts less than about 5 wt %) can be beneficial for achieving the target properties of the nanofibers and/or formulations derived therefrom. In a specific example, the nanofibers do not include a significant amount of hemicellulose and/or lignin. In another example, the cellulose can include trace amounts of alginate (e.g., 0.1-5% of the total weight of the nanofiber can be alginate), where the alginate can be beneficial for improving dispersion of the nanofiber.

In a first variant, the nanofiber(s) 100 are preferably substantially composed of cellulose (e.g., 95-99.9999 wt %, or values contained therebetween, cellulose). Each nanofiber can be a single cellulose polymer chain but alternatively can be made up of a plurality of cellulose polymer chains (e.g., forming microfibrils, etc.). In variants in which the nanofiber is made up of a plurality of cellulose polymer chains, the cellulose chains can be held together via physical entanglement, hydrogen bonds, dipole-dipole interactions, Van der Waal interactions, polymeric crosslinking, and/or any other suitable physical or chemical interaction. In these variants, the cellulose may form a linear fiber, a branched fiber, or any other structure. In these variants, individual cellulose chains can be aligned with each other or misaligned. However, the nanofiber can be otherwise structured.

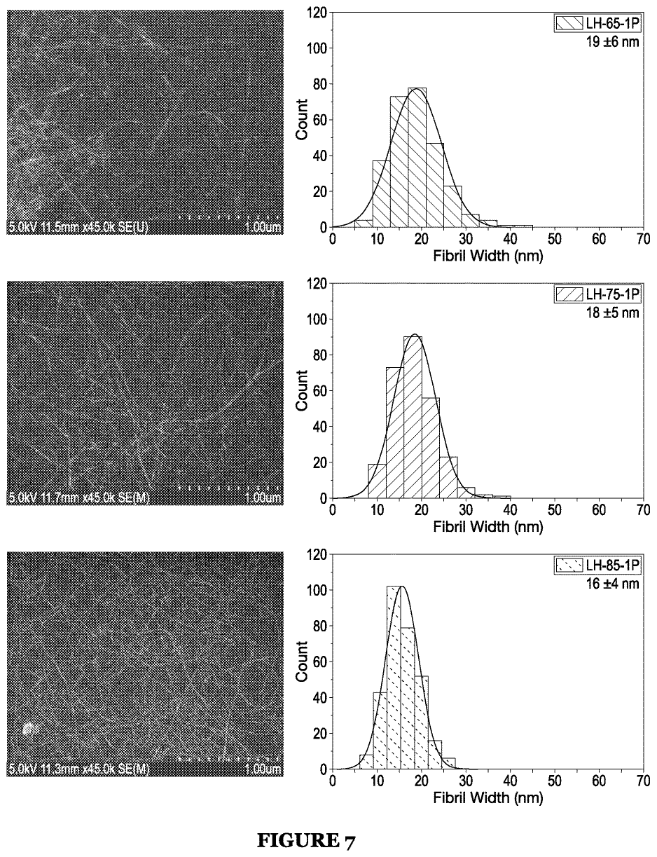

The nanofiber 100 preferably has a thickness (e.g., width, extent in a direct orthogonal to a longest axis, etc.) between 0.1 and 100 nm (e.g., 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, or any value therebetween), as shown for example in FIG. 7. The thickness of the nanofiber can depend on the manufacturing process, the origin plant (e.g., seaweed) species, growing conditions of the plant (e.g., seaweed) species, age of the harvested plant (e.g., seaweed), and/or other suitable characteristics of the plant(s) and/or manufacturing process. In a specific example, the nanofiber can have an average thickness between 1 and 10 nm. The nanofiber can have a uniform thickness across its length and/or can be variable (e.g. bumpy, fibrous, textured along the length of the fiber, etc.) and/or can have any other thickness variation. The nanofiber can have a cross-sectional shape that is substantially ovate (e.g., circular, elliptical, etc.), amorphous, and/or any other shape.

The nanofiber 100 can have a length between about 500 nm and 1000 micron (e.g., 490 nm, 500 nm, 600 nm, 700 nm, 800 nm, 900 nm, 1 micron, 2 micron, 3 microns, 4 micron, 5 micron, 6 micron, 7 micron, 8 micron, 9 micron, 10 micron, 20 micron, 30 micron, 40 micron, 50 micron, 60 micron, 70 micron, 80 micron, 90 micron, 100 micron, 200 μm, 300 μm, 400 μm, 500 μm, 600 μm, 750 μm, 800 μm, 900 μm, 1 mm, 1.05 mm, a value therebetween, etc.). The length of the nanofiber can depend on the manufacturing process, the origin plant species, growing conditions of the plant species, age of the harvested plant, and/or other suitable characteristics of the plant(s) and/or manufacturing process.

The dimensions of the nanofiber 100 can be characterized based an aspect ratio between the thickness and length. The nanofiber preferably has a length that is between about 300 and 10000 times larger than its thickness (e.g., 275:1, 300:1, 350:1, 400:1, 450:1, 500:1, 550:1, 600:1, 650:1, 700:1, 750:1, 800:1, 850:1, 900:1, 950:1, 1000:1, 1500:1, 2000:1, 2500:1, 3000:1, 3500:1, 4000:1, 5000:1, 7500:1, 10000:1, and/or any value or range therebetween). In contrast, wood cellulose can achieve an aspect ratio at most about 100:1. In some variants, the aspect ratio and/or distribution of aspect ratios (e.g., aspect ratio standard deviation, aspect ratio variance, number of modes of the distribution, skew of the distribution, distribution shape, etc.) within a formulation is understood to be critical for achieving specific performance (e.g., target rheological properties) and, as such, may need to be tuned for different applications. For instance, a first aspect ratio and/or aspect ratio distribution can be favorable for spray on sunscreens while a second aspect ratio and/or aspect ratio distribution can be favorable for creams and/or lotions. However, in other variants, surface charge, length, width, composition, modification, ratio of modified to unmodified nanofibers, and/or other properties can additionally or alternatively be critical. In some variants, the aspect ratio of nanofibers can be characterized based on statistical averages due to natural variation in fiber length produced during fibrillation processes. For example, while the average diameter of nanofibers can be relatively consistent (e.g., approximately 9-10 nm), nanofiber lengths can exhibit a distribution of values due to the manufacturing process.

In specific variants, the nanofibers 100 can exhibit high aspect ratios and extended fiber lengths. For example, fiber widths can be on the order of tens of nanometers, while fiber lengths can extend to several micrometers or greater. In a specific example, the nanofibers can have an average length greater than 300 times the average thickness. The resulting aspect ratios can exceed those observed for cellulose nanofibrils derived from wood sources. The long lengths can promote physical entanglement and network formation within aqueous suspensions, which can contribute to the rheological properties and suspension stability of the nanofiber dispersions.

The nanofiber 100 preferably has a negative surface charge, but can alternatively have a neutral surface and/or a positive surface charge. The negative surface charge can result from functional groups on the surface of the nanofiber (e.g., hydroxyl, carboxyl, sulfonic, phosphoric, nitro, silicate, borate, etc. functional group(s)). For example, acetic acid, oxalic acid, citric acid, malic acid, tartaric acid, and/or lactic acid can be derivatized onto a surface of the cellulosic nanofibers (and subsequently deprotonated such as using a basic solution or high pH to result in a negative surface charge). Additionally or alternatively, the negative surface charge can result from species sorbed to the nanofiber, from defects and/or dangling bonds of the nanofiber, a pH of the solution (e.g., formulation), and/or from other sources. Similarly, a positive surface charge can result from functionalization of the nanofibers (e.g., resulting from functionalization of the nanofibers such as with amine groups, based on the formulation or additives thereof, solution pH, etc.). In some variants, the magnitude surface charge of the nanofiber(s) can be the native surface charge magnitude and/or can be modified (e.g., increased or decreased such as via surface functionalization reactions such as using a process as described in U.S. patent application Ser. No. 18/833,792 titled “Method for producing surface modified nanocellulose material” filed 3 Feb. 2023 which is incorporated in its entirety by this reference).

In some variants, a surface charge density of the nanofiber can be a critical parameter influencing the rheological behavior and functional performance of nanofiber dispersions. Surface charge density can refer to the density of charged functional groups present on a surface of the nanofiber (e.g., charges per unit surface area or per unit mass of nanofiber, percentage and/or fraction of chargeable sites that are charged and/or functionalized, etc.). The inventors have found that varying the surface charge density of the nanofibers can significantly alter rheological characteristics such as viscosity, yield stress, shear stress response, shear strain response, storage modulus, loss modulus, and/or suspension stability. For example, nanofibers having relatively low surface charge density (e.g., substantially unmodified nanofibers) can exhibit different dispersion and rheological behavior compared to nanofibers having moderate or high surface charge densities (e.g., nanofibers that have been surface functionalized to introduce charged functional groups). In some variants, the surface charge density can be tuned across a range (e.g., between approximately 0% and 100% of a target functionalization level, or any value therebetween, etc.), which can enable customization of rheological properties for specific applications. The inventors have found that increased charged density (e.g., negative charge) can cause fibers to swell more in aqueous suspensions and/or dispersions. In variants, the inventors have found that nanofibers with a surface charge density between 15% and 30% can result in formulations that enable adequate suspension. The inventors have additionally found that nanofibers with a charge density of 100% has a high storage modulus with reasonable flow modulus, indicating good suspension and texture properties, as shown for example in FIG. 11B. However, the nanofiber can have any charge density to achieve any rheological properties.

In variants, the nanofibers can include charged functional groups (e.g., anionic functional groups, carboxyl groups, carboxylate groups, cationic functional groups, quaternary ammoniums, etc.) on a surface of the nanofibers. In examples, the nanofibers can include surface charged functional groups at an average density between to approximately 0 and 5000 micromoles/g (e.g., 0 micromoles/g, 100 micromoles/g, 200 micromoles/g, 300 micromoles/g, 400 micromoles/g, 500 micromoles/g, 600 micromoles/g, 700 micromoles/g, 800 micromoles/g, 900 micromoles/g, 1000 micromoles/g, 2000 micromoles/g, 3000 micromoles/g, 4000 micromoles/g, 5000 micromoles/g, or any value and/or range therebetween), such as at a pH between approximately 3 and 10. In variants, the charged functional groups can be introduced through oxidation processes (e.g., (2,2,6,6-Tetramethylpiperidin-1-yl)oxyl (TEMPO)-mediated oxidation), chemical functionalization, and/or any other suitable surface modification process. In variants, the nanofibers can have a TEMPO-oxidation density (corresponding to a carboxylate group densities) between approximately 0 micromoles/g and 5000 micromoles/g (e.g., 0 micromoles/g, 100 micromoles/g, 200 micromoles/g, 300 micromoles/g, 400 micromoles/g, 500 micromoles/g, 600 micromoles/g, 700 micromoles/g, 800 micromoles/g, 900 micromoles/g, 1000 micromoles/g, 2000 micromoles/g, 3000 micromoles/g, 4000 micromoles/g, 5000 micromoles/g, or any value and/or range therebetween.), such as at a pH between approximately 3 and 10. In some variants, the oxidation density can be selected to tune a balance between suspension performance, viscosity, texture, yield stress, and/or flow behavior for different formulations and applications. However, the surface charge density can be otherwise defined.

The properties of the nanofiber can contribute (e.g., influence, be selected based on, etc.) to various behavioral and rheological properties (e.g., rheological behavior as characterized in FIGURE. 4). For instance, the nanofiber can have high water absorption and particle suspension capabilities that can make it suitable for retaining and releasing bioactives. The capabilities of the nanofiber can be characterized from a release profile, degradation rate, and/or other suitable measurements.

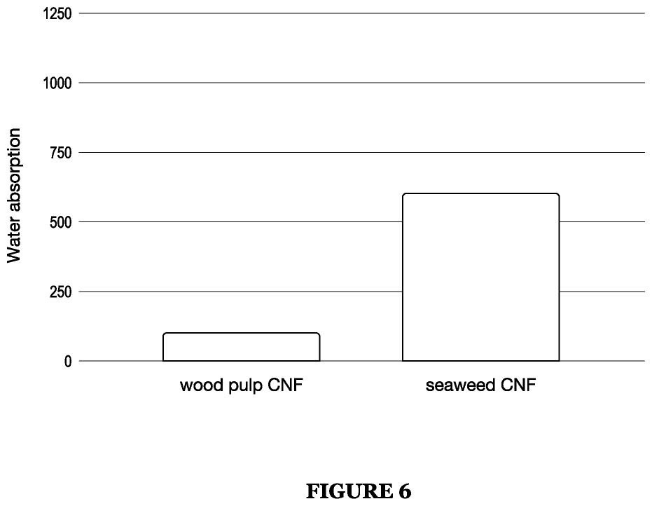

The nanofiber 100 can exhibit a high-water absorption capacity and/or water holding capacity. The nanofiber's water capacity can be quantified by:

$$WC = \frac{{mass}_{hydrated}}{{mass}_{dehydrated}} ,$$

where WC refers to the water capacity (water absorption capacity, water holding capacity), mass_{hydrated} describes the weight of water the nanofiber retains (e.g. absorbs, holds, etc.), and mass_{dehydrated} described the dry weight of the nanofibers. The nanofibers can have a water capacity between 100 and 1000 or any range or value therebetween (e.g., 100, 200, 300, 400, 500, 600, 700, 800, 900, 1000), as shown for example in FIG. 6. That is, the nanofibers can retain an amount of water between approximately 100 and 1000 times its weight. In a first variant, the nanofibers can have a high density of hydroxyl groups (e.g., natively, resulting from surface functionalization, etc.) which can result in hydrophilic nanofibers. In a second variant, a high aspect ratio can contribute to high water absorption by resulting in a high surface area that provides an increased number of sites for water molecules to adhere to (e.g., via hydrogen bonding). In a third variant, the nanofibers can form a 3D matrix (e.g., hydrogel, etc.) that can hold water through capillary action and/or physical entrapment. In a fourth variant, the nanofibers can have and/or form a porous structure that traps water. In a fifth variant, the nanofibers can combine any or all of the first through fourth variants.

In some variants, the water absorption capacity of the nanofiber can correspond to swelling of the nanofiber structure. For example, the nanofiber can swell upon hydration such that the nanofiber structure can absorb and retain water at levels up to approximately between 100 and 700 times its dry mass. However, the nanofiber can swell to levels lower than 100 times its dry mass or above 700 times its dry mass. In a specific variant, the nanofiber swells to approximated 500 times its dry mass. This swelling can be facilitated by the high surface area of the nanofibers, hydrogen bonding interactions between water molecules and hydroxyl groups of cellulose, and/or electrostatic interactions associated with charged functional groups on the nanofiber surface. The degree of swelling can further depend on the surface charge density of the nanofiber, the density of hydrophilic functional groups present on the nanofiber surface, the aspect ratio, and/or any other properties.

The nanofiber(s) can be dispersible and/or suspensible in a solvent. The nanofiber can remain suspended in solution for at least 5 hours without significant precipitate formation (e.g., 6 hours, 8 hours, 12 hours, 24 hours, 36 hours, 48 hours, 72 hours, 96 hours, 1 week, 2 weeks, 1 month, 2 months, 4 months, 6 months, 8 months, 9 months, 12 months, indefinitely, etc.).

The nanofiber 100 can allow for particle suspension. Additionally or alternatively, the nanofiber can facilitate or improve suspension of chemical species (e.g., additives as described below, active ingredients, inactive ingredients, etc.). The nanofiber and particle or chemical species can remain suspended in solution for at least 5 hours without showing signs of precipitate formation (e.g., 6 hours, 8 hours, 12 hours, 24 hours, 36 hours, 48 hours, 72 hours, 96 hours, 1 week, 2 weeks, 1 month, 2 months, 4 months, 6 months, 8 months, 9 months, 12 months, indefinitely, etc.).

The nanofiber's ability to suspend species can be dependent on its elastic modulus, yield stress, the particle mass, nanofiber charge, particle charge, molecular structure, and/or other characteristics. In a first example, a nanofiber with a higher elastic modulus and/or yield stress can suspend heavier particles by resisting deformation. In a second example, a negative surface charge on the nanofibers can favorably interact with positively surface charged particles (e.g., ZnO, TiO₂, etc.). In a third example, the nanofiber can create a 3D matrix that can entrap particles.

The nanofiber can have a yield stress between 0.01-100 Pa or any range or value therebetween (e.g. 0.01 Pa, 0.05 Pa, 0.1 Pa, 0.2 Pa, 0.3 Pa, 0.4 Pa, 0.5 Pa, 0.6 Pa, 0.7 Pa, 0.8 Pa, 0.9 Pa, 1 Pa, 2 Pa, 3 Pa, 4 Pa, 5 Pa, 6 Pa, 7 Pa, 8 Pa, 9 Pa, 10 Pa, 20 Pa, 30 Pa, 40 Pa, 50 Pa, 60 Pa, 70 Pa, 80 Pa, 90 Pa, 100 Pa, or any value therebetween). The yield stress can alternatively be less than 0.01 Pa or greater than 100 Pa.

The nanofiber 100 can have an elastic modulus between 0.01 Pa and 1 MPa (e.g. 0.01 Pa, 0.05 Pa, 0.1 Pa, 0.5 Pa, 1 Pa, 5 Pa, 10 Pa, 50 Pa, 100 Pa, 1000 Pa, 5000 Pa, 10000 Pa, 100000 Pa, 1 MPa, or any value therebetween). The elastic modulus can alternatively be less than 0.01 Pa or greater than 1 MPa.

As shown for example in FIG. 4, the nanofibers can have an elastic modulus that is between 1-50000 times larger than its loss modulus or any range or value therebetween (e.g., 2, 3, 4, 10, 50, 75, 100, 200, 500, 1000, 2000, 5000, 10000, 20000, 50000, values or ranges therebetween, etc.) across frequencies and/or shear strain during rheological testing. The elastic modulus and loss modulus can alternatively be otherwise related. The nanofiber can have any other mechanical properties.

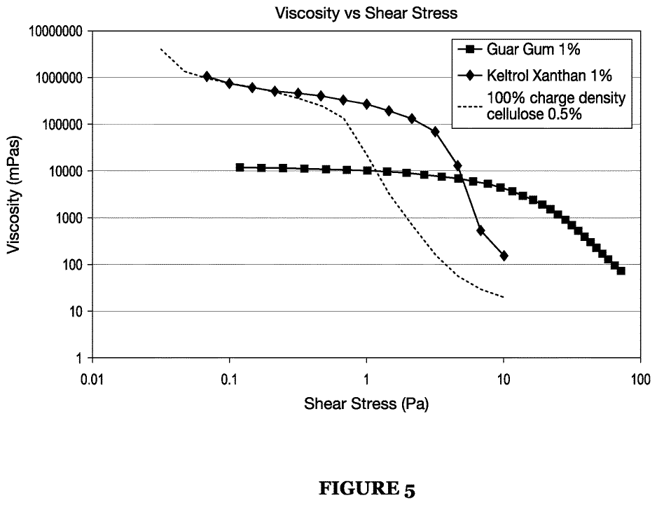

The nanofiber 100 can exhibit distinct rheological behaviors in dispersions (e.g., formulation, samples made for rheological testing, etc.). In one preferred example, the nanofiber exhibited shear thinning behavior (e.g., becoming less viscous with increasing applied shear stress, as shown for example in FIG. 5). This shear thinning behavior can be exhibited as a linear decrease on a log-log plot of viscosity vs shear rate, as shown for example in FIG. 4. The nanofiber's shear thinning behavior can allow and/or enable nanofiber utilization in formulations dispensed from a nozzle, pump, atomizer, needle, and/or other similar delivery mechanisms. In an example, the nanofiber can be added to spray-on sunscreens or spray-on hair products (e.g. shine spray, texturizing spray, spray tan, etc.). Relatedly, the low shear stress can result in nanofiber-containing formulations that feel non-viscous, thin, and/or waterlike at low applied shear stresses. The low shear stress and shear thinning behavior can allow the nanofiber to have a pleasant, non-sticky skin feel when topically applied. However, the nanofiber can exhibit other rheological behaviors (e.g., based on the surface modification, nanofiber concentration, etc.).

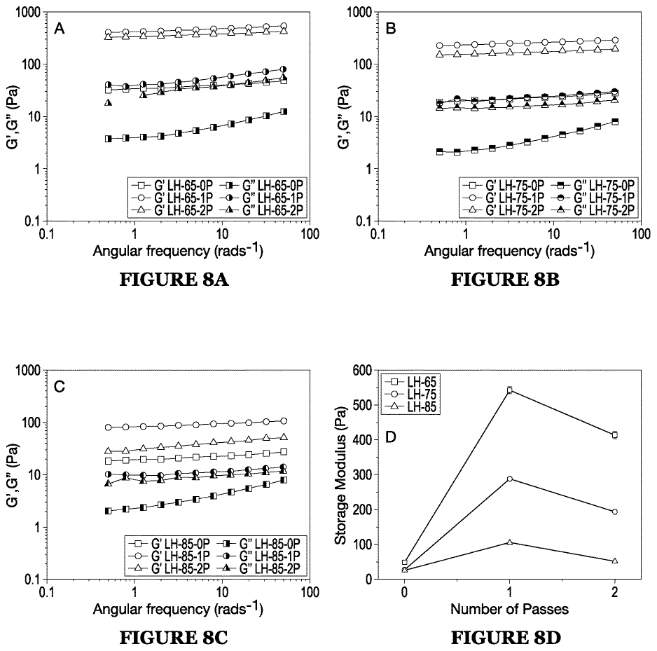

In some variants, aqueous suspensions of the nanofibers can exhibit gel-like viscoelastic behavior. For example, rheological measurements can show storage modulus (G′) values greater than loss modulus (G″) across a range of frequencies, and the storage modulus can be relatively independent of angular frequency, as shown for example in FIG. 8. The viscoelastic properties can arise from a combination of electrostatic repulsion between charged fibril surfaces and attractive intermolecular forces, which together produce a physically entangled fiber network within the suspension. In some variants, aqueous dispersions of the nanofibers can exhibit viscoelastic behavior even at lower nanofiber concentrations. For example, nanofiber suspensions at concentrations of approximately 0.25 wt % to 5 wt % (0.25%, 0.5%, 1%, 2%, 3%, 4%, 5%, and/or any value therebetween) can exhibit viscosities greater than that of water and can form structured dispersions or gel-like materials. In illustrative examples, nanofiber suspensions at approximately 1 wt % concentration can exhibit storage modulus (G′) values on the order of tens to hundreds of pascals, such as approximately 40 Pa to 600 Pa or more depending on processing conditions and degree of functionalization. In some variants, the viscosity and viscoelastic properties of the dispersion can depend on factors such as nanofiber concentration, aspect ratio, surface charge density, and degree of fibrillation.

In some variants, surface charge density can directly influence rheological properties of nanofiber dispersions. For example, increasing the surface charge density of the nanofibers can increase electrostatic repulsion between adjacent fibers, which can affect fiber swelling, dispersion stability, and network formation within a suspension. In some variants, increased surface charge density can promote swelling of the nanofibers and modify rheological parameters such as viscosity, yield stress, and viscoelastic moduli. Conversely, reduced surface charge density can result in different fiber aggregation behavior and altered rheological responses. As such, surface charge density can function as a tunable parameter for designing nanofiber dispersions with specific rheological performance characteristics suitable for particular formulations (e.g., sprayable formulations, creams, gels, suspensions, etc.).

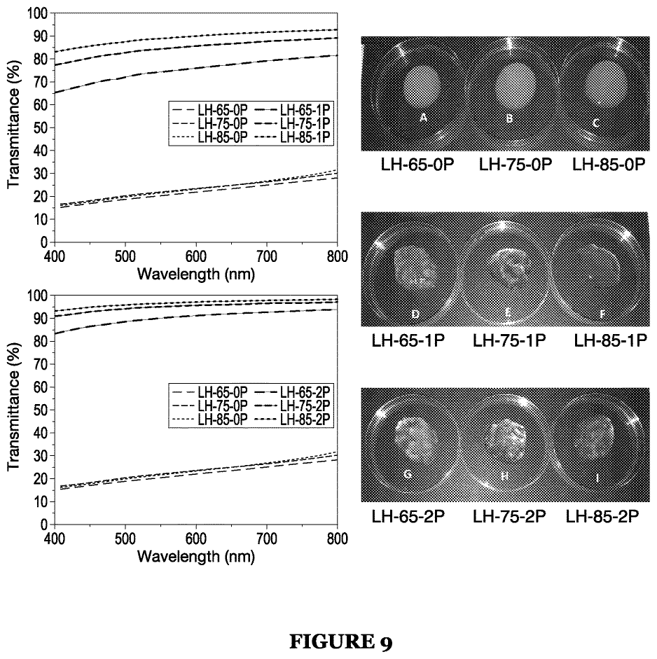

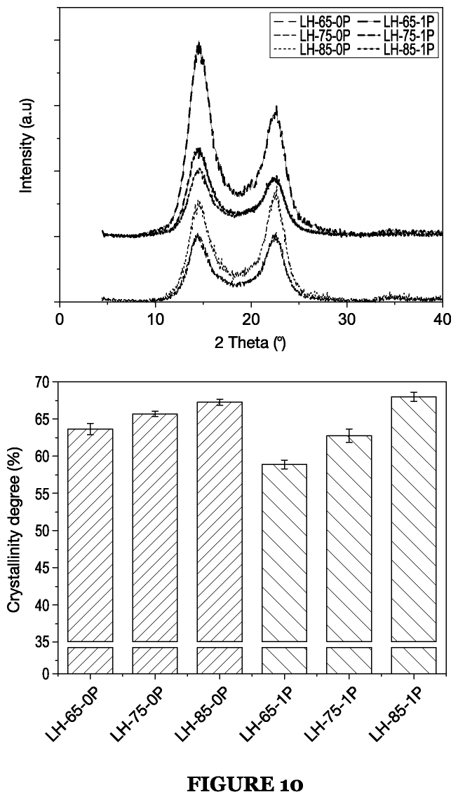

In some variants, the nanofibers can exhibit relatively high crystallinity values. For example, cellulose derived from macroalgae sources can exhibit crystallinity indices of between 50% to 70%, as shown for example in FIG. 10, depending on extraction and processing conditions. In some variants, the dispersions and/or suspensions of the nanofibers can have transmittance between 60% and 100% depending on the processing conditions, as shown for example in FIG. 9.

The nanofibers 100 can be stable in various environments and conditions. In a first variant, the nanofiber can be stable (e.g. non-reactive, resistant to degradation, resistant to oxidation, etc.) in high temperatures. For example, the nanofiber can remain stable in temperatures between −200 °C and 400 °C (e.g. −200 °C, −100 °C, 0 °C, 10 °C, 20 °C, 30 °C, 40 °C, 50 °C, 60 °C, 70 °C, 80 °C, 90 °C, 100 °C, 200 °C, 300 °C, 400 °C or any value or range therebetween). In a second variant, the nanofiber can be stable in high and/or low PH levels. For example, the nanofiber can be stable in pH levels between 3 and 12 (e.g. 3, 3.5, 4, 4.5, 5, 5.5, 6, 6.5, 7, 7.5, 8, 8.5, 9, 9.5, 10, 10.5, 11, 11.5, 12, or any value or range therebetween). In a third variant, the nanofiber can be stable when mixed in solution with oxides (e.g., zinc oxide, titanium dioxide, iron oxides, magnesium oxide, etc.), electrolytes, and/or salts (e.g. sodium PCA, sodium lactate, magnesium sulfate, sodium chloride, sodium bicarbonate, sodium hydroxide, potassium hydroxide, sodium salicylate, sea salt, Himalayan salt, zinc sulfate, etc.). In a fourth variant, the nanofibers can be stabile to the aforementioned conditions (e.g., combinations of two or more of heat, pH, and the presence of salt or oxides) concurrently. The nanofiber can exhibit stability in other conditions and/or can be further improved by the inclusion of antioxidants (or anti-degradation species) within the formulation.

The nanofibers can be manufactured using mechanical fibrillation methods (e.g., grinding, ultra sonification, high-pressure homogenization, microfluidization, etc.), chemical methods (e.g., acid hydrolysis, carboxymethylation, enzymatic hydrolysis, TEMPO-mediated oxidation, etc.), and/or any suitable methods (e.g., as described in U.S. patent application Ser. No. 17/293,655 titled “Methods for processing fibrous cellulosic material, products and uses thereof” filed 13 Nov. 2019 which is incorporated in its entirety by this reference).

The nanofibers can optionally be modified. Modified typically refers to surface functionalization. However, modified can additionally or alternatively include mechanical processing (e.g., to modify a width and/or length of the nanofiber), chemical processing, physical processing (e.g., temperature, pressure), and/or other suitable processes. Typically, the modified nanofibers and unmodified nanofibers will have different physical properties (e.g., width, length, aspect ratio, surface charge magnitude, surface charge sign, etc.). As a specific example, modified nanofibers can have a larger aspect ratio the unmodified nanofibers (e.g., because modification can result in improved detanglement of fibers within a bundle). As another specific example, modified nanofibers can have a greater (e.g., in magnitude) surface charge than unmodified nanofibers. However, one or more of the physical properties can be the same between the modified and unmodified nanofibers.

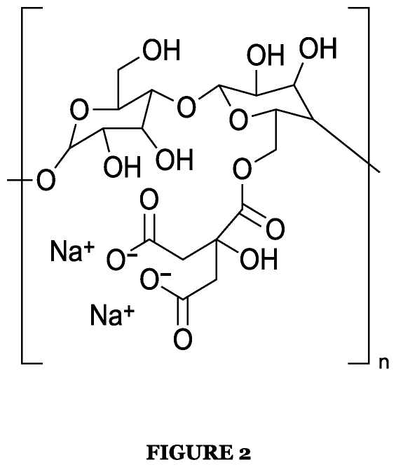

In one specific example, the nanofiber can be processed to have different surface functionalization (e.g., as described in U.S. patent application Ser. No. 18/833,792 titled “Method for producing surface modified nanocellulose material” filed 3 Feb. 2023 which is incorporated in its entirety by this reference). In a variant, hydroxyl groups of the nanofiber can be modified to decrease hydrophilicity and/or alter the nanofiber to be hydrophobic. In an example, surface modification can be achieved through silylation, acetylation, esterification, oxidation, etherification, carboxylation, sulfonation, phosphorylation, amination, silanization, or any suitable method. In variants, the nanofibers can be modified with oxidizing agents (e.g., TEMPO-mediated oxidation, sodium periodate, ammonium persulfate, nitric acid oxidation, etc.), carboxylic acids (e.g., citric acid as shown for example in FIG. 2, oxalic acid, malic acid, succinic acid, tartaric acid, maleic acid, etc.), silanes (e.g., trimethoxysilanes, APTES, octyltriethoxysilane, etc.), anhydrides (e.g., acetic anhydride, propionic anhydride, butyric anhydride, etc.), sulfating or phosphorylating agents (e.g., sulfuric acid, chlorosulfonic acid, phosphoric acid, etc.), amines (e.g., ethylenediamine, polyethyleneimine, quaternary ammonium compounds, etc.), polymer grafts (e.g., PEG, PLA, PAA, PNIPAM, etc.), or nanoparticles (e.g., silver, gold, TiO₂, ZnO, etc.), or combinations thereof. The hydroxyl groups of a nanofiber can be all modified or partially modified. Partial modification can result in a nanofiber being amphiphilic (e.g., allowing it to function as an emulsifier). In other variants, the nanofiber can be functionalized to increase reactivity, increase biocompatibility, and/or add functionality (e.g. grafting responsive polymers for pharmaceutical or topical treatment). In an example, functional groups such as —CH₃, —NH₂, —SH, alkyl chains, PEG chains, PLA chains, PAA chains, —OH, —SiOₓ, and/or —COOH, can be added to the nanofiber to alter its functionalization. In some variants, surface functionalization can introduce charged functional groups and thereby alter a surface charge density of the nanofiber, which can influence the rheological properties and suspension capability of the formulation.

In a first variant, the formulations can include nanofibers that have not been modified. In a second variant, the formulations can include essentially only nanofibers that have been modified. In a third variant, nanofiber formulations can include a combination of nanofibers that have been surface functionalized and nanofibers that have not been surface functionalized. The ratio of modified and unmodified nanofibers can be altered to achieve desirable physical properties in product formulation (e.g., physical properties as described above), to achieve a target aspect ratio distribution, and/or to achieve other target properties. A nanofiber blend preferably has a ratio (e.g., weight ratio, atomic ratio, stoichiometric ratio, volume ratio, etc.) of unmodified to modified nanofibers between 1:10 to 10:1 (e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, or any suitable ratio). Alternatively, a nanofiber blend can be characterized by a weight percentage and/or volume percentage of unmodified and/or modified nanofibers (e.g. 10% modified, 15% modified, 20% modified, 25% modified, 30% modified, 35% modified, 40% modified, 45% modified, 50% modified, 60% modified, 70% modified, 80% modified, 90% modified, any value therebetween, etc.). Various mixtures of functionalized and non-functionalized nanofiber can be preferable for increased suspension or emulsification properties, but can additionally and/or alternatively be used for other properties.

The nanofiber(s) can be dispersible and suspensible in a solvent 200. The solvents can act as a carrier for the nanofibers and/or additives. While typically a liquid, the solvent can be a solid matrix (e.g., petrolatum). In some variants, the solvent(s) can serve an additive function as well (e.g., ethanol can act as a solvent and disinfectant). Examples of solvents include water, alcohols (e.g., ethanol, isopropyl alcohol, etc.), oils (e.g., fatty acids, triglycerides, etc.), ketones (e.g., acetone), esters (e.g., ethyl acetate), combinations thereof, and/or other suitable solvent(s) can be used. The nanofiber(s) preferably disperse within the solvent(s) (particularly but not exclusively water, ethanol, isopropyl alcohol) with minimal shear (e.g., using low shear mixers, low shear impellers, planetary mixers, conical screw mixers, etc.).

However, in some variants, the nanofibers (and optionally additives) can be provided as a powder (e.g., substantially dry, with entrapped water or other solvent, lyophilized powder, etc. such as with particle sizes between 50 and 2000 μm).

In a formulation, the concentration of nanofibers can depend on the final form of the formulation (e.g., lotion, cream, gel, liquid, paste, etc.). Typically, the formulation includes between about 0.1 wt % and 7 wt % (e.g., 0.09 wt %, 0.1 wt %, 0.2 wt %, 0.3 wt %, 0.5 wt %, 0.75 wt %, 1 wt %, 1.1 wt %, 1.2 wt %, 1.3 wt %, 1.5 wt %, 1.6 wt %, 1.75 wt %, 1.9 wt %, 2 wt %, 2.25 wt %, 2.3 wt %, 3 wt %, 3.3 wt %, 3.7 wt %, 4 wt %, 4.2 wt %, 4.5 wt %, 5 wt %, 5.15 wt %, 5.6 wt %, 6 wt %, 6.3 wt %, 6.6 wt %, 7 wt %, 7.2 wt %, values or ranges therebetween, etc.) total of nanofibers.

The additives 300 function to modify one or more physical and/or chemical behavior of the formulation (and/or nanofibers themselves). The additives can include oils (e.g., mineral oil, coconut oil, jojoba oil, argan oil, sunflower oil, castor oil, balsam, etc.), emollients (e.g. caprylic/capric triglyceride, isopropyl myristate, shea butter, lanolin, ceramides, etc.), occlusives (e.g., beeswax, carnauba wax, paraffin wax, petrolatum, candelilla wax, etc.), emulsifiers (e.g., polysorbates, lecithin, PEG derivatives, etc.), humectants (e.g., glycerin, propylene glycol, butylene glycol, sorbitol, hyaluronic acid, etc.), stabilizers (e.g., xanthan gum, carbomer, acrylates copolymers, etc.), mica, pigments (e.g., iron oxides, titanium dioxide, ultramarines, etc.), dyes (e.g., FD&C colors, etc.), minerals (e.g., zinc oxide, kaolin clay, bentonite, magnesium oxide, cerium oxide, sulfur, etc.), abrasive particles (e.g., silica, pumice, walnut shell powder, capsules, microbeads, etc.), UV blockers (e.g., zinc oxide, titanium oxide, p-aminobenzoic acid, padimate, phenylbenzimidazole sulfonic acid, cinoxate, dioxybenzone, oxybenzone, homosalate, menthyl anthranilate, octocrylene, octinoxate, octyl salicylate, sulisobenzone, avobenzone, ecamsule, etc.), preservatives (e.g., phenoxyethanol, parabens, benzyl alcohol, ethylhexylglycerin, etc.), antioxidants (e.g., vitamin E [tocopherol], ascorbic acid, green tea extract, etc.), surfactants (e.g., sodium lauryl sulfate, coco-glucoside, decyl glucoside, etc.), film formers (e.g., polyvinyl alcohol, acrylates or methacrylate monomers or polymers, etc.), conditioning agents (e.g., panthenol, quaternary ammonium compounds, etc.), fragrances and essential oils, pH adjusters (e.g., citric acid, sodium hydroxide, triethanolamine, etc.), chelating agents (e.g., EDTA, phytic acid, etc.), thickeners (e.g., cellulose derivatives, guar gum, alginates, etc.), polymers (e.g., PVP, HPMC, acrylates, etc.), enzymes (e.g., protease, lipase, cellulase, etc.), natural extracts (e.g., aloe vera, chamomile, calendula, witch hazel, etc.), proteins & peptides (e.g., silk protein, collagen, peptides for anti-aging, etc.), salts (e.g., sodium chloride, magnesium sulfate, etc.), silicones (e.g., cyclopentasiloxane, dimethicone, etc.), antimicrobials (e.g. antibiotics, antifungals, antiseptics, etc.), anti-inflammatory agents (e.g., corticosteroids, anti-allergens, etc.), dermatological actives (e.g. anti-acne agents, calamine, colloidal oatmeal, allantoin, bisabol, benzoyl peroxide, azelaic acid, niacinamide, lactic acid, mandelic acid, salicylic acid, retinol, etc.), and/or other suitable ingredients. Additives can be included in relatively small amounts (e.g., less than about 1 wt % per additive). However, in some variants, larger amounts of additives can be used (as one such example diaper creams and/or sunscreens can include zinc oxide particles in concentrations of 10-30 wt %, but many other examples and/or concentrations can be realized). However, the nanofibers can be combined with other materials using any suitable post-processing method.

In variants in which the formulation includes particles and/or insoluble material, the nanofibers can function as dispersants or suspension agents for insoluble materials. For example, nanofiber dispersions can stabilize suspensions of particulate materials such as mineral particles, pigments, active pharmaceutical ingredients, or other insoluble compounds. The fibrillar network formed by the nanofibers is hypothesized to hinder or prevent sedimentation of such particles by increasing viscosity and forming a structured suspension matrix; however, alternative explanations may explain or contribute to the suspension properties. In illustrative examples, nanofiber dispersions can suspend insoluble particulate materials (e.g., calcium carbonate) within aqueous formulations. In some variants, the nanofibers enable suspension of relatively high concentrations of particulate material at low nanofiber loadings. For example, stable suspensions can be achieved with nanofiber concentrations of about 0.05 wt % to 0.5 wt % while maintaining particle concentrations between 1 wt % and 50 wt %, depending on particle size and density. In illustrative examples, an aqueous dispersion comprising about 0.2 wt % nanofibers can stably suspend about 10 wt % of oxide particles (e.g., zinc oxide, titanium dioxide, etc.). In variants, the nanofiber can form suspensions with particle-to-nanofiber weight ratios between 1:50 and 50:1 without substantial sedimentation over a relevant storage period. However, higher or lower loadings may be achieved depending on formulation conditions.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the preceding system and/or method can be used with, in addition to, in lieu of, or otherwise integrated with all or a portion of the systems and/or methods disclosed in the applications mentioned above, each of which are incorporated in their entirety by this reference.

As used herein, “substantially” or other words of approximation (e.g., “about,” “approximately,” etc.) can be within a predetermined error threshold or tolerance of a metric, component, or other reference (e.g., within 0.001%, 0.01%, 0.1%, 1%, 5%, 10%, 20%, 30% of a reference), or be otherwise interpreted.

As a person skilled in the art will recognize from the previous detailed description and from the figures and claims, modifications and changes can be made to the preferred embodiments of the invention without departing from the scope of this invention defined in the following claims.

We claim:

1. A formulation comprising:
an aqueous dispersion of seaweed-derived nanofibers, wherein the seaweed-derived nanofibers consist essentially of cellulose, wherein the aqueous dispersion comprises between 0.05% and 2% of seaweed-derived nanofibers by weight, wherein the seaweed-derived nanofibers have a water uptake greater than 500× a dry weight of the seaweed-derived nanofibers, wherein the aqueous dispersion has a viscosity that decreases with increasing shear rate.

2. The formulation of claim 1, wherein the aqueous dispersion has a storage modulus that is greater than a loss modulus across two magnitudes of frequency.

3. The formulation of claim 1, wherein an average diameter of the seaweed-derived nanofibers is between 7 and 12 nm.

4. The formulation of claim 1, wherein an average length of the seaweed-derived nanofibers is greater than 300 times an average diameter of the seaweed-derived nanofibers.

5. The formulation of claim 1, wherein the seaweed-derived nanofibers comprise surface charged functional groups up to an average density of 500 micromoles/g, at a pH between 6 and 9.

6. The formulation of claim 1, wherein the seaweed-derived nanofibers comprise a TEMPO-oxidization density for surface charged functional groups between 500 micromoles/g and 1700 micromoles/g, at a pH between 6 and 9.

7. The formulation of claim 1, wherein the seaweed-derived nanofibers comprise modified nanofibers and non-modified fibers, wherein a ratio of modified nanofibers to unmodified nanofibers is between 1:10 and 10:1, wherein the modified fibers comprise a greater density of charged surface groups than the unmodified fibers at a pH between 4 and 12.

8. The formulation of claim 7, wherein the modified fibers comprise carboxylate functional groups bonded to surface hydroxyl groups and wherein surface hydroxyl groups of the unmodified fibers do not substantially comprise carboxylate functional groups.

9. The formulation of claim 1, wherein the formulation is for at least one of: lotion, cream, gel, serum, paste, or aerosol.

10. A nanocellulose fiber comprising:
an average diameter that is between 1 and 12 nm;
an average length greater than 300 times the average diameter; and
a negative surface charge density of 20%-50%;
wherein the nanocellulose fiber is derived from seaweed.

11. The nanocellulose fiber of claim 10, wherein a water swelling capacity of the nanocellulose fiber is greater than 500× a dry weight of the nanofiber.

12. The nanocellulose fiber of claim 10, wherein the nanocellulose fiber comprises cellulose in an amount greater than 95 wt %.

13. The nanocellulose fiber of claim 10, wherein the nanocellulose fiber comprises less than 5 wt % hemicellulose and lignin.

14. The nanocellulose fiber of claim 10, wherein the nanocellulose fiber comprises alginate in an amount between 0.1 wt % and 5 wt %.

15. The nanocellulose fiber of claim 10, wherein the nanocellulose fiber forms a shear-thinning aqueous dispersion at a concentration of less than 1 wt %.

16. The nanocellulose fiber of claim 10, wherein the nanocellulose fiber comprises a crystallinity index between 50% and 95%.

17. A formulation comprising:
a plurality of the nanocellulose fibers of claim 10;
a solvent; and
a plurality of particles suspended within a continuous phase comprising the solvent and the plurality of nanocellulose fibers.

18. The formulation of claim 17, wherein a concentration of nanocellulose fibers is between 0.05 wt % and 0.75 wt % of the formulation and a concentration of particles is between 5 wt % and 40 wt %.

19. The formulation of claim 17, wherein the formulation further comprises at least one an oil, an emollient, a humectant, an emulsifier, a pigment, a preservative, or a UV blocker.

20. The formulation of claim 17, wherein the plurality of particles comprises zinc oxide or titanium dioxide.
